(19) United States
(12) Plant Patent Application Publication
(10) Pub. No.: US 2003/0106118 P1
(43) Pub. Date: Jun. 5, 2003

(54) Hybrid tea rose plant named 'Miss Amber'

(76) Inventor: James W. Coiner, La Verne, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/015,905

(22) Filed: Dec. 5, 2001

(51) Int. Cl. A01H 5/00

(52) U.S. Cl. PLT/139

(57) ABSTRACT

A new and distinct Hybrid Tea Rose plant variety with orange flowers.

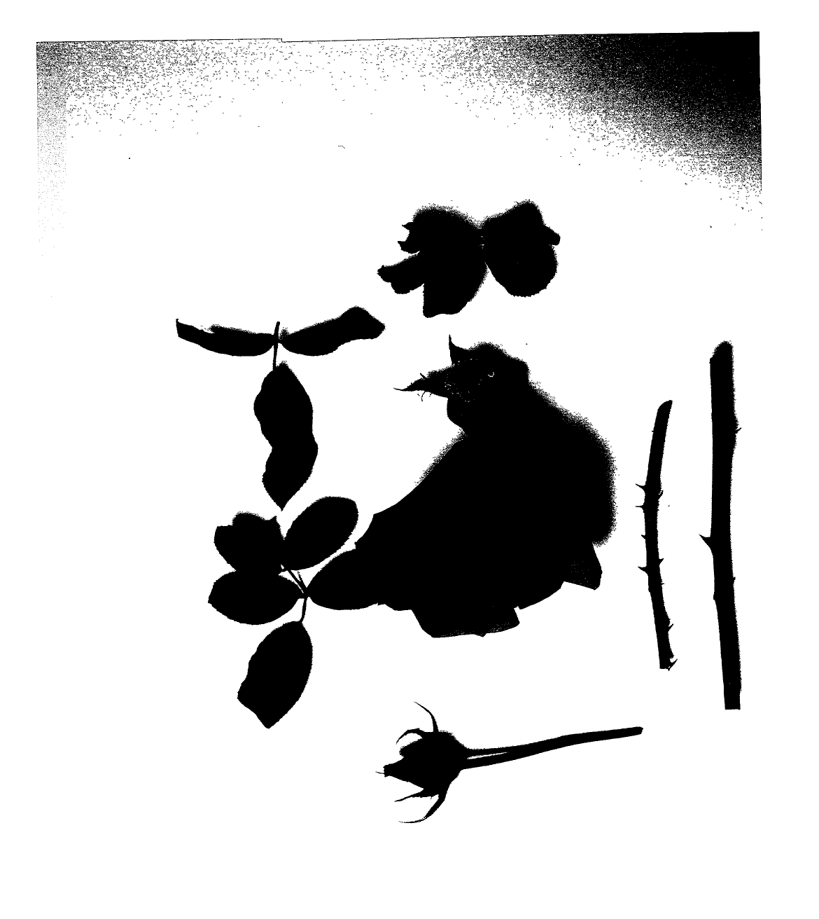

### BACKGROUND OF THE INVENTION

The present invention relates to a new and distinct variety of rose plant of the hybrid tea rose which was created by crossing as seed and pollen parents two (2) unknown rose varieties. The varietal denomination of the new variety is ‘MISS AMBER’.

### SUMMARY OF THE INVENTION

Among the novel characteristics possessed by this new variety which distinguishes it from other varieties of which I am aware, is its orange flowers.

Asexual reproduction by propagation by budding of the new variety as performed in Wasco, Calif. shows that the foregoing and other distinguishing characteristics come true to form and are established and transmitted through succeeding propagations.

### BRIEF DESCRIPTION OF ILLUSTRATION

The accompanying illustration shows typical specimens of the flowers of the new variety depicted in color as nearly true as it is reasonably possible to make the same in a color illustration of this character.

### DESCRIPTION OF THE NEW VARIETY

The following is a detailed description of 2-year budded plants of the new variety , with color terminology in accordance with The Royal Horticultural Society Colour Chart (RHSCC). The terminology used in color description herein refers to plate numbers in the aforementioned color chart. Phenotypic expression may vary with light availability, environmental and cultural conditions.

The following observations are made of specimens grown outdoors in Wasco, Calif., during November.

### PLANT

Form: Spreading

Growth: Semi-vigorous

Size: Height about 36 inches; width about 36 inches

Foliage:

Size.—Length about 2¼ inches; width about 1½ inches.

Quantity.—About 5.

Color.—New foliage: Upper side near 178A; under side near 179A Mature Upper side near 137A; under foliage: side near 137D.

Shape.—Elliptic.

Texture.—Upper side: Smooth Under side: Matte.

Edge.—Serrated.

Serration.—Serrate.

Petioles.—Top surface color near 143B; Under side near 143C.

Stipules.—Medium length.

Rachis.—Few prickles.

Variegation.—None.

Veination.—None.

### WOOD

New shoots:

Bark.—Smooth.

Wood.—Near 178B.

Old wood:

Bark.—Smooth.

Wood.—Near 143A.

Flowering stem: Length about 3 inches

Winter Hardiness: Completely winter hardy

Resistance to Disease: Fair

Temperature range: Between 16° and 120° F.

Preferred growing conditions: Full sun

Growth habit: Medium vigor

Leaves per stem: About 5

### FLOWER/INFLORESCENCE

Blooming Habit: Continuous

Bud.—Length about 2 inches; width about 1 9/16 inches. Form: Classic rose form Color: Before sepals divide color near 33A; when sepals first divide color near 33A; when petals begin to unfurl color near 37A

Sepals.—Long and thin Size: Length in relation to bud, about 1½ inches Color Topside near 143B; bottom near 144A.

Peduncle.—Length: About 3 inches Strength: Erect Color: Near 178B

Flower:

Size.—About 5¼ inches.

Borne.—Single.

Form.—High center; unfurls full.

Petalage: About 26

Petaloids in center.—About 7.

Persistence.—Good.

Fragrance: Slight

Lasting Quality: About 3 weeks on plant; about 7 days after cutting

Color:

During first 2 days.—Inner petals at flower center topside near 37A; bottom near 38A; outer petals topside near 37B, bottom near 38B.

Base of petals (Point of attachment): Near 2A

General tonality from a distance: Near 38B

Color Change: Remains the same even after 3 or more days

Petals:

Texture.—Thin/soft.

Shape.—Longer than wider.

Arrangement.—Imbricated.

Appearance.—Velvety.

### REPRODUCTIVE ORGANS

Arrangement of stamens: Regularly arranged around

Filaments and Anthers: styles

Color: Filaments near 7A; Anthers near 10A

Pollen: No pollen

Styles: Thin; medium length

Stigmas: Usual

Hips/Seed Vessels/Ovaries:

Shape.—Round.

Size.—Small; about ¼ inch.

Color.—Near 138B.

Seeds: No seeds

I claim:

1. A new and distinct Hybrid Tea Rose plant of the variety substantially as shown and described.
